(19) United States
(12) Patent Application Publication
(10) Pub. No.: US 2026/0108342 A1
(43) Pub. Date: Apr. 23, 2026

(54) IMPLANTABLE BODY ARMOR FOR PROTECTING INTERNAL ORGANS

(71) Applicant: BALLAST MEDICAL INC., Montreal (CA)

(72) Inventor: Michel GAGNER, Montreal (CA)

(21) Appl. No.: 19/176,694

(22) Filed: Apr. 11, 2025

Related U.S. Application Data
(60) Provisional application No. 63/634,022, filed on Apr. 15, 2024.

(51) Int. Cl. A61F 2/02; A61L 27/18; A61L 27/54; A61L 27/58

(52) U.S. Cl. CPC A61F 2/02; A61L 27/18; A61L 27/54; A61L 27/58; A61F 2210/0004; A61F 2210/0076; A61F 2250/0067; A61F 2310/0097

(57) ABSTRACT

An implantable body armor for placement at an implantation site within a ventral cavity of a human body is provided. The implantable body armor includes a flexible panel having an intercostal surface and a cavity surface opposite the intercostal surface, the flexible panel including layers of woven fibers forming an impact-resistant structure configured to absorb impact energy. The flexible panel can be further configurable between a delivery configuration and a functional configuration wherein, when in the delivery configuration, the flexible panel has an external surface area that is smaller than when in the functional configuration to facilitate a delivery of the implantable body armor to the implantation site.

### CROSS-REFERENCE TO RELATED APPLICATION

The present application claims priority from U.S. provisional patent application No. 63/634,022, filed on Apr. 15, 2024, entitled “IMPLANTABLE BODY ARMOR FOR PROTECTING INTERNAL ORGANS”, the disclosure of which is hereby incorporated by reference in its entirety.

### TECHNICAL FIELD

The technical field generally relates to implants for implantation within a human body. In particular, the technical field relates to an implantable body armor for the protection of internal organs.

### BACKGROUND

Conventional body armors and other protective devices can be used by law enforcement personnel, security forces, military, and the like, for protection against severe injury or death from bullets, knives, shrapnel, and other projectiles and sharp objects. Such protective devices can be configured to stop a bullet or other projectile or sharp from entering the torso or other portion of a body of a person, but require that a wearer dons the protective device externally as a piece of clothing to benefit from the potential protection it can provide.

However, protective devices are generally bulky and cumbersome to wear, which can reduce the comfort and mobility of the wearer. Moreover, the size of the protective devices typically renders them difficult to conceal under regular clothing, while providing an unpleasant aesthetic appearance.

Accordingly, there remain a number of challenges with respect to traditional body armors and other protective devices.

### SUMMARY

In accordance with an aspect, there is provided an implantable body armor for placement at an implantation site within a ventral cavity of a human body, the implantable body armor comprising a flexible panel having an intercostal surface and a cavity surface opposite the intercostal surface, the flexible panel comprising layers of woven fibers forming an impact-resistant structure configured to absorb impact energy.

In some implementations, the cavity surface has a cavity surface roughness that is higher than an intercostal surface roughness of the intercostal surface.

In some implementations, the intercostal surface is substantially smooth.

In some implementations, the implantable body armor further comprises an implant frame engageable with the flexible panel and positionable around an outer periphery of the flexible panel.

In some implementations, the implant frame is a self-deployable implant frame configured to transition the implantable body armor from a delivery configuration to a functional configuration once the implantable body armor is delivered at the implantation site.

In some implementations, the self-deployable implant frame is configured to transition to a deployed configuration once delivered at the implantation site.

In some implementations, when the self-deployable implant frame is configured in the deployed configuration at the implantation site, the flexible panel is configured for placement against an internal organ outer surface.

In some implementations, the self-deployable implant frame comprises one or more resilient members configured to bias the implantable body armor from the delivery configuration to the functional configuration.

In some implementations, the implant frame is made of an implant frame material, the implant frame material being bioabsorbable.

In some implementations, the implant frame or the flexible panel comprises an attachment feature to releasably connect the implantable body armor to a delivery system during delivery of the implantable body armor.

In some implementations, the delivery system is a laparoscopic tool or a thoracoscopic tool.

In some implementations, the implantable body armor further comprises a fixation mechanism to engage the flexible panel with a hard tissue or a soft tissue located in the vicinity of the implantation site.

In some implementations, the fixation mechanism is configured to engage the flexible panel with an internal surface of a thoracic cage.

In some implementations, the fixation mechanism is made of a fixation mechanism material, the fixation mechanism material being bioabsorbable.

In some implementations, adjacent fibers of the layers of woven fibers define interstices sized to promote a growth of tissue therebetween to contribute to an integration of the flexible panel into an adjoining tissue.

In some implementations, an outermost layer of the layers of woven fibers comprises a first interstice zone defining first zone interstices having a first size selected to promote a growth of tissue therebetween, and a second interstice zone defining second zone interstices having a second size to prevent a growth of tissue therebetween, the second size being smaller than the first size.

In some implementations, one of the first and second interstice zones of the outermost layer extends over a central region of the outermost layer, and the other of the first and second interstice zones of the outermost layer extends over a peripheral region of the outermost layer extending peripherally around the central region.

In some implementations, a given one of the first and second interstice zones comprises discontinued regions spaced apart from each other and distributed over a surface area of the outermost layer of the layers of woven fibers.

In some implementations, the woven fibers of adjacent ones of the layers of woven fibers are superposed and provided in a cross-layered arrangement.

In some implementations, adjacent ones of the layers of woven fibers are bound together using one or more of a resin and an adhesive to form a laminated flexible panel.

In some implementations, the resin comprises one or more of a polyurethane-based resin, an epoxy resin, and a thermoplastic resin.

In some implementations, fibers of the layers of woven fibers comprise aramid fibers.

In some implementations, the aramid fibers comprise para-aramid fibers.

In some implementations, the para-aramid fibers comprise Kevlar™ fibers.

In some implementations, fibers of the layers of woven fibers comprise ultra high molecular weight polyethylene fibers.

In some implementations, the ultra high molecular weight polyethylene fibers comprise Dyneema™ fibers.

In some implementations, the fibers of the layers of woven fibers have a density of less than 1.5 g/cm³.

In some implementations, at least one layer of the layers of woven fibers further comprises additional fibers.

In some implementations, the additional fibers comprise bioabsorbable fibers absorbable at physiological conditions.

In some implementations, the additional fibers have hydrophobic properties to prevent bacterial adhesion in an interstice defined between adjacent fibers of the layers of woven fibers.

In some implementations, at least a portion of the additional fibers comprises a bioactive agent having therapeutic properties.

In some implementations, the layers of woven fibers are configured as a plurality of superposed woven layers.

In some implementations, the plurality of superposed woven layers comprises at least 3 superposed woven layers.

In some implementations, the plurality of superposed woven layers comprises at least 6 superposed woven layers.

In some implementations, the implantable body armor further comprises a panel cover at least partially covering the flexible panel, the panel cover being made of a biocompatible material.

In some implementations, the biocompatible material comprises a bioactive agent having therapeutic properties.

In some implementations, outermost layers of the layers of woven fibers comprise a biocompatible coating provided onto an external surface thereof.

In some implementations, the biocompatible coating comprises a bioactive agent having therapeutic properties.

In some implementations, the bioactive agent comprises at least one of a cell growth promoter, a cell growth inhibitor, an antibiotic, a cytokine, a healing promoter, a clotting modulator, an anti-inflammatory, and an anti-scarring agent.

In some implementations, the flexible panel is configured to resist a penetration of a projectile or a pointed object to protect an internal organ.

In some implementations, the flexible panel comprises a reinforcement zone provided at a selected location of the flexible panel to provide an additional impact-resistant structure.

In some implementations, the reinforcement zone comprises at least one of additional layers of woven fibers, a metal armor, and a ceramic armor.

In some implementations, the metal armor comprises at least one of MIL-A-46100 steel, AR500 steel, titanium and aluminum.

In some implementations, the ceramic armor comprises at least one of boron carbide and silicon carbide.

In some implementations, the reinforcement zone is engaged with the cavity surface or the outer surface of the flexible panel.

In some implementations, the reinforcement zone is provided between adjacent ones of the layers of woven fibers of the flexible panel.

In accordance with another aspect, there is provided an implantable body armor for placement at an implantation site within a dorsal cavity of a human body, the implantable body armor comprising a flexible panel having an outer surface and a cavity surface opposite the outer surface, the flexible panel comprising layers of woven fibers forming an impact-resistant structure configured to absorb impact energy.

In some implementations, the implantable body armor is implantable in a cranial cavity of the dorsal cavity.

In some implementations, the implantable body armors is implantable in a spinal cavity of the dorsal cavity.

In some implementations, the implantable body armor further comprises one or more features as defined above.

In accordance with another aspect, there is provided an implantable body armor for placement at an implantation site within a human body, the implantable body armor comprising a flexible panel comprising layers of woven fibers forming an impact-resistant structure configured to absorb impact energy, wherein the implantable body armor is configurable between a delivery configuration and a functional configuration, wherein when in the delivery configuration, the flexible panel of the implantable body armor has a resulting external surface area that is smaller than when in the functional configuration to facilitate a delivery of the implantable body armor to the implantation site.

In some implementations, the flexible panel is rollable onto itself to form a flexible panel roll to place the implantable body armor in the delivery configuration.

In some implementations, the flexible panel is foldable onto itself to place the implantable body armor in the delivery configuration.

In some implementations, the flexible panel comprises a folding line provided at a predetermined location to facilitate a transition of the implantable body armor between the delivery configuration and the functional configuration.

In some implementations, the flexible panel comprises a reinforcement zone to provide an additional impact-resistant structure.

In some implementations, the reinforcement zone comprises first and second reinforcement zones located in first and second selected regions, respectively, of the layers of woven fibers, the folding line extending between the first and second reinforcement zones.

In some implementations, when in the delivery configuration, the implantable body armor is deliverable to the implantation site through an intercostal space of a thoracic cage.

In some implementations, when in the delivery configuration, the implantable body armor is deliverable to the implantation site through a thoracic cage.

In some implementations, the implantable body armor further comprises an implant frame engageable with the flexible panel and positionable around an outer periphery of the flexible panel.

In some implementations, the implant frame is a self-deployable implant frame configured to transition the implantable body armor from the delivery configuration to the functional configuration once the implantable body armor is delivered at the implantation site.

In some implementations, the self-deployable implant frame is configured to transition to a deployed configuration once delivered at the implantation site.

In some implementations, when the self-deployable implant frame is configured in the deployed configuration at the implantation site, the flexible panel is configured for placement against an internal organ outer surface.

In some implementations, the self-deployable implant frame comprises one or more resilient members configured to bias the implantable body armor from the delivery configuration to the functional configuration.

In some implementations, the implant frame comprises a bioabsorbable material.

In some implementations, the implant frame or the flexible panel comprises an attachment feature to releasably connect the implantable body armor to a delivery system during delivery of the implantable body armor.

In some implementations, the delivery system is one of a laparoscopic tool and a thoracoscopic tool.

In some implementations, the implantable body armor further comprises a fixation mechanism to engage the flexible panel with a hard tissue or a soft tissue located in the vicinity of the implantation site.

In some implementations, the fixation mechanism is configured to engage the flexible panel with an internal surface of a thoracic cage.

In some implementations, adjacent fibers of the layers of woven fibers define interstices sized to promote a growth of tissue therebetween to contribute to an integration of the flexible panel into an adjoining tissue.

In some implementations, fibers of the layers of woven fibers comprise aramid fibers.

In some implementations, the aramid fibers comprise para-aramid fibers.

In some implementations, the para-aramid fibers comprise Kevlar™ fibers.

In some implementations, fibers of the layers of woven fibers comprise ultra high molecular weight polyethylene fibers.

In some implementations, the ultra high molecular weight polyethylene fibers comprise Dyneema™ fibers.

In some implementations, the fibers of the layers of woven fibers have a density of less than 1.5 g/cm³.

In some implementations, at least one layer of the layers of woven fibers further comprises additional fibers.

In some implementations, the additional fibers comprise bioabsorbable fibers absorbable at physiological conditions.

In some implementations, the additional fibers have hydrophobic properties to prevent bacterial adhesion in an interstice defined between adjacent fibers of the layers of woven fibers.

In some implementations, the additional fibers comprise a bioactive agent having therapeutic properties.

In some implementations, the layers of woven fibers are configured as a plurality of superposed woven layers.

In some implementations, the plurality of superposed woven layers comprises at least 3 superposed woven layers.

In some implementations, the plurality of superposed woven layers comprises at least 6 superposed woven layers.

In some implementations, the implantable body armor further comprises a panel cover at least partially covering the flexible panel, the panel cover being made of a biocompatible material.

In some implementations, the biocompatible material comprises a bioactive agent having therapeutic properties.

In some implementations, outermost layers of the layers of woven fibers comprise a biocompatible coating provided onto an external surface thereof.

In some implementations, the biocompatible coating comprises a bioactive agent having therapeutic properties.

In some implementations, the bioactive agent comprises at least one of a cell growth promoter, a cell growth inhibitor, an antibiotic, a cytokine, a healing promoter, a clotting modulator, an anti-inflammatory, and an anti-scarring agent.

In some implementations, the flexible panel is configured to resist a penetration of a projectile or a pointed object to protect an internal organ.

In some implementations, the flexible panel comprises a cavity surface and an outer surface opposite the cavity surface.

In some implementations, the outer surface is an intercostal surface.

In some implementations, a given one of the cavity surface and the outer surface has a reduced coefficient of friction relative to the other one of the cavity surface and the outer surface, the given one of the cavity surface and the outer surface forming the resulting external surface of the implantable body armor when the implantable body armor is in the delivery configuration.

In accordance with another aspect, there is provided a method for delivering an implantable body armor at an implantation site, the method comprising delivering the implantable body armor to an implantation site in a delivery configuration, the implantable body armor comprising a flexible panel comprising layers of woven fibers forming an impact-resistant structure configured to absorb impact energy, and transitioning the implantable body armor from the delivery configuration to a functional configuration once the implantable body armor is positioned at the implantation site to provide the impact resistance to an internal organ, the delivery configuration being different than the functional configuration.

In some implementations, delivering the implantable body armor comprises laparoscopically delivering the implantable body armor to the implantation site.

In some implementations, laparoscopically delivering the implantable body armor comprises delivering the implantable body armor through a percutaneous incision to a subcutaneous implantation site or a submuscular implantation site.

In some implementations, laparoscopically delivering the implantable body armor comprises delivering the implantable body armor through an abdominal wall, and optionally through a peritoneum and into an abdominopelvic cavity.

In some implementations, delivering the implantable body armor comprises thoracospically delivering the implantable body armor to the implantation site.

In some implementations, thoracospically delivering the implantable body armor comprises delivering the implantable body armor through an intercostal space of a thoracic cage.

In some implementations, delivering the implantable body armor further comprises releasably connecting an attachment feature of the implantable body armor to a delivery system.

In some implementations, the method further comprises releasing the attachment feature to enable a deployment of the flexible panel into the functional configuration.

In some implementations, the method further comprises fastening the implantable body armor to one or more of a soft tissue and a hard tissue in the vicinity of the implantation site.

In some implementations, fastening the implantable body armor comprises fastening the implantable body armor to an internal surface of a thoracic cage.

In some implementations, the method further comprises promoting a growth of soft tissue or hard tissue within interstices defined by adjacent fibers of the woven fibers of the layers of woven fibers when the implantable body armor is in the functional configuration to contribute to an integration of the implantable body armor into an adjoining tissue.

In accordance with another aspect, there is provided an implantable body armor for placement at an implantation site within a human body, the implantable body armor comprising a flexible panel comprising layers of woven fibers forming an impact-resistant structure configured to absorb impact energy, wherein the implantable body armor is configurable between a delivery configuration and a functional configuration, wherein when the flexible panel is in the delivery configuration, a transversal width and a height of the flexible panel provide a first aspect ratio TW:H sized in accordance with a cross-section of a delivery path of the implantable body armor within the human body, and when the flexible panel is in the functional configuration, the transversal width and the height of the flexible panel provide a second aspect ratio different than the first aspect ratio.

In some implementations, the flexible panel is rollable onto itself to form a flexible panel roll to place the implantable body armor in the delivery configuration.

In some implementations, the flexible panel is foldable onto itself to place the implantable body armor in the delivery configuration.

In some implementations, the flexible panel comprises one or more folding lines extending at corresponding predetermined locations to facilitate a transition of the implantable body armor between the delivery configuration and the functional configuration.

In some implementations, the flexible panel comprises a reinforcement zone to provide an additional impact-resistant structure.

In some implementations, the reinforcement zone comprises multiple reinforcement zones located in selected regions of the layers of woven fibers, at least one of the one or more folding lines extending between adjacent ones of the reinforcement zones.

In some implementations, when the flexible panel is in the functional configuration, the first aspect ratio TW:H is between about 4:1 and about 20:1, between about 10:1 and about 30:1, or between about 15:1 and about 50:1.

In some implementations, when the flexible panel is in the delivery configuration, the second aspect ratio of about 1:1, between about 0.75:1 and about 1.5:1, between about 0.5:1 and about 2:1, or between about 1.5:1 and about 3:1.

In some implementations, when in the delivery configuration, the implantable body armor is deliverable to the implantation site through an intercostal space of a thoracic cage.

In some implementations, when in the delivery configuration, the implantable body armor is deliverable to the implantation site through a thoracic cage.

In some implementations, the implantable body armor further comprises an implant frame engageable with the flexible panel and positionable around an outer periphery of the flexible panel.

In some implementations, the implant frame is a self-deployable implant frame configured to transition the implantable body armor from the delivery configuration to the functional configuration once the implantable body armor is delivered at the implantation site.

In some implementations, the self-deployable implant frame is configured to transition to a deployed configuration once delivered at the implantation site.

In some implementations, when the self-deployable implant frame is configured in the deployed configuration at the implantation site, the flexible panel is configured for placement against an internal organ outer surface.

In some implementations, the self-deployable implant frame comprises one or more resilient members configured to bias the implantable body armor from the delivery configuration to the functional configuration.

In some implementations, the implant frame comprises a bioabsorbable material.

In some implementations, the implant frame or the flexible panel comprises an attachment feature to releasably connect the implantable body armor to a delivery system during delivery of the implantable body armor.

In some implementations, the delivery system is one of a laparoscopic tool and a thoracoscopic tool.

In some implementations, the implantable body armor further comprises a fixation mechanism to engage the flexible panel with a hard tissue or a soft tissue located in the vicinity of the implantation site.

In some implementations, the fixation mechanism is configured to engage the flexible panel with an internal surface of a thoracic cage.

In some implementations, adjacent fibers of the layers of woven fibers define interstices sized to promote a growth of tissue therebetween to contribute to an integration of the flexible panel into an adjoining tissue.

In some implementations, fibers of the layers of woven fibers comprise aramid fibers.

In some implementations, the aramid fibers comprise para-aramid fibers.

In some implementations, the para-aramid fibers comprise Kevlar™ fibers.

In some implementations, fibers of the layers of woven fibers comprise ultra high molecular weight polyethylene fibers.

In some implementations, the ultra high molecular weight polyethylene fibers comprise Dyneema™ fibers.

In some implementations, the fibers of the layers of woven fibers have a density of less than 1.5 g/cm3.

In some implementations, at least one layer of the layers of woven fibers further comprises additional fibers.

In some implementations, the additional fibers comprise bioabsorbable fibers absorbable at physiological conditions.

In some implementations, the additional fibers have hydrophobic properties to prevent bacterial adhesion in an interstice defined between adjacent fibers of the layers of woven fibers.

In some implementations, the additional fibers comprise a bioactive agent having therapeutic properties.

In some implementations, the layers of woven fibers are configured as a plurality of superposed woven layers.

In some implementations, the plurality of superposed woven layers comprises at least 3 superposed woven layers.

In some implementations, the plurality of superposed woven layers comprises at least 6 superposed woven layers.

In some implementations, the implantable body armor further comprises a panel cover at least partially covering the flexible panel, the panel cover being made of a biocompatible material.

In some implementations, the biocompatible material comprises a bioactive agent having therapeutic properties.

In some implementations, outermost layers of the layers of woven fibers comprise a biocompatible coating provided onto an external surface thereof.

In some implementations, the biocompatible coating comprises a bioactive agent having therapeutic properties.

In some implementations, the bioactive agent comprises at least one of a cell growth promoter, a cell growth inhibitor, an antibiotic, a cytokine, a healing promoter, a clotting modulator, an anti-inflammatory, and an anti-scarring agent.

In some implementations, the flexible panel is configured to resist a penetration of a projectile or a pointed object to protect an internal organ.

In some implementations, the flexible panel comprises a cavity surface and an outer surface opposite the cavity surface.

In some implementations, the outer surface is an intercostal surface.

In some implementations, a given one of the cavity surface and the outer surface has a reduced coefficient of friction relative to the other one of the cavity surface and the outer surface, the given one of the cavity surface and the outer surface forming an external surface of the implantable body armor when the implantable body armor is in the delivery configuration.

### BRIEF DESCRIPTION OF THE DRAWINGS

The attached figures illustrate various features, aspects and implementations of the technology described herein.

FIG. 1 is a drawing of an upper human body illustrating the various body cavities thereof.

FIG. 2 is an exploded top perspective view of an implantable body armor, in accordance with an implementation, shown in a functional configuration.

FIG. 3 is a top perspective view of an implantable body armor, in accordance with another implementation, shown in a functional configuration.

FIG. 4 is a cross-sectional view of the implantable body armor of FIG. 2.

FIG. 5 is an exploded top perspective view of an implantable body armor, in accordance with another implementation.

FIG. 6 is a top perspective view of the implantable body armor of FIG. 2, shown in a transitional state between the functional configuration and a delivery configuration.

FIG. 7 is a top perspective view of the implantable body armor of FIG. 2, shown in a delivery configuration.

FIG. 8 is a top perspective view of the implantable body armor, in accordance with another implementation, shown in a delivery configuration.

FIG. 9 is a top perspective view of the implantable body armor of FIG. 8, shown in a functional configuration.

FIG. 10 is a top perspective view of an implantable body armor, in accordance with yet another implementation.

### DETAILED DESCRIPTION

Techniques described herein relate to implantable body armors that can be implanted into a cavity of a human body to reduce the risk of injury to an internal organ contained therein. The implantable body armor includes a flexible panel comprising layers of woven fibers forming an impact-resistant structure having desirable properties. Examples of desirable properties can include a high strength-to-weight ratio, a high tensile strength, a high tear strength and abrasion resistance, and other high energy absorption characteristics, while being thin, flexible and lightweight. In some implementations, the impact-resistant structure can also be configured to prevent penetration of sharp objects, such as bladed weapons, therethrough. Providing an implantable body armor having these properties can provide various benefits including facilitating implantation of the implantable body armor within said cavity of the human body and enabling a higher level of comfort following implantation of the implantable body armor.

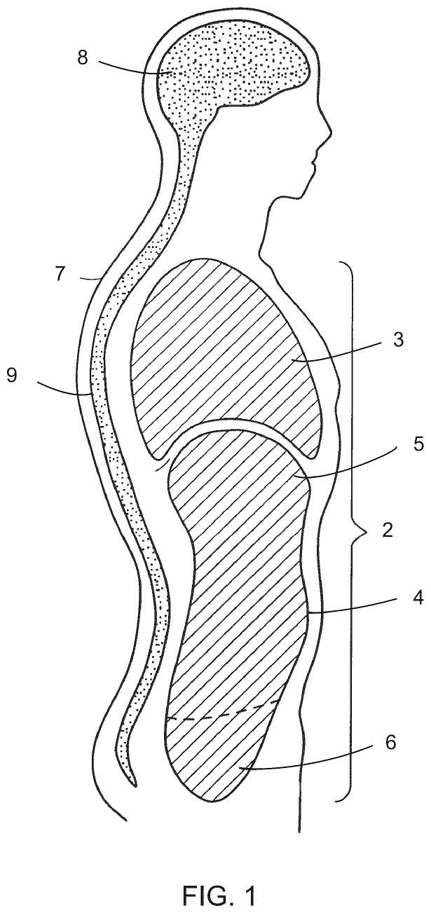

The desirable properties mentioned above can provide an implantable body armor suitable for implantation at various locations within the human body, such as within one of multiple cavities of the human body, which are schematically represented in FIG. 1. For instance, the implantable body armor can be suitable for implantation within one of the ventral cavity 2 and the dorsal cavity 7. The ventral cavity 2 is bound by the rib cage and the abdominal musculature and includes the thoracic cavity 3 and the abdominopelvic cavity 4. The thoracic cavity 3 is separated from the abdominopelvic cavity 4 by the diaphragm. The abdominopelvic cavity 4 is below the diaphragm, and includes the abdominal cavity 5 and the pelvic cavity 6. The dorsal cavity 7 includes the cranial cavity 8 housing the brain, and the spinal cavity 9 housing the spinal cord.

The thoracic cavity 3 and the abdominopelvic cavity 4 define anatomic potential spaces having a dynamic volume capable of increasing in volume (i.e., a space between two adjacent anatomical structures that are normally pressed together and/or directly apposed). For instance, the thoracic cavity 3 includes the pleural cavity defined between opposing layers of the pulmonary pleurae which separates the lungs from the rib cage and the mediastinum, as well as the pericardial cavity defined between the fibrous pericardium and the serous pericardium surrounding the heart. The abdominopelvic cavity 4 includes the peritoneal cavity surrounding the abdominal organs and defined between the parietal peritoneum and the visceral peritoneum, within the abdomen.

In some implementations, the features and the properties of the implantable body armor, as described in greater detail below, can enable an implantation of the implantable body armor within a cavity of the human body. For instance, the implantable body armor can be implanted within the thoracic cavity 3 or within the peritoneal cavity. In some implementations, when the implantable body armor is implanted in the thoracic cavity 3, the implantable body armor can be positioned against the ribs of the rib cage, in back of the ribs (i.e., between the ribs and the parietal pleura), or within the pleural cavity, for example. In some implementations, when the implantable body armor is implanted in the abdominopelvic cavity 4, the implantable body armor can be positioned within the peritoneal cavity or against the parietal peritoneum, for example. It is to be understood that in other implementations, the implantable body armor can alternatively be implantable in front of the ribs, i.e., between the skin or superficial fascia and the ribs, or subcutaneously, at the level of the abdomen, or internally against the abdominal wall.

The properties of the implantable body armor can further enable providing a resulting implantable body armor having a distinct technical profile that can make it suitable for implantation within one of the cavities of the human body described above in proximity of an internal organ or a group of internal organs. For instance, in some implementations, the implantable body armor can be implanted in the vicinity of an internal organ undergoing periodic motion, such as the heart or the lung as they undergo periodic contraction and expansion without interfering with the proper physiological functioning of the internal organ or the group of internal organs. It is to be understood that, in other implementations, the implantable body armor can be implanted in proximity of any other internal organ to provide protection thereto including, for instance, an artery, a vein, a nerve, the spinal cord, the brain (including, for instance, the cerebellum), the neck (including, for instance, cervical structures), the liver, the stomach, the kidney, and the pancreas.

It will be appreciated that the implantation of the implantable body armor within one of the cavities of the human body described above can provide a lasting concealed form of protection to internal organs. For example, the implantation of the implantable body armor within a cavity of the human body (in contrast to, for instance, a subcutaneous or submuscular placement of the implantable body armor) can substantially separate the implantable body armor from the muscular system. In some implementations, the separation of the implantable body armor from the muscular system can reduce the risk of undesirable interference between the implantable body armor and a muscle during muscle contraction or relaxation and/or interference introduced following the reshaping of muscles brought on by changes in activity levels or the aging process.

While the description of the implantable body armor presented above relates to the implantation of the implantable body armor within a cavity of the human body, it is to be understood that, in other implementations, the desirable properties mentioned above can provide an implantable body armor suitable for a subcutaneous implantation or submuscular implantation within the human body (i.e., at a location outside of the ventral cavity 2 and the dorsal cavity 7). The implantation of the implantable body armor at a subcutaneous implantation site or a submuscular implantation site may be desirable to provide a protection to an internal organ by means of a less-invasive surgical procedure. For instance, in some implementations, the implantable body armor can be inserted at a submuscular implantation site between a muscle and an adjacent fascia layer, i.e., the implantable body armor can be sandwiched between a muscle and an adjacent fascia layer. In other implementations, the implantable body armor can be implanted at a subcutaneous implantation site, such as in proximity of the vertebrae to provide protection to the spinal cord, among other examples.

In some implementations, when the implantable body armor is configured for a subcutaneous implantation or a submuscular implantation within the human body, the properties of the implantable body armor can make it suitable for extending over a greater area compared to the implantable body armor when configured for implantation within a cavity of the human body. For instance, in some implementations, the implantable body armor can be configured for implantation at a subcutaneous or submuscular implantation site to provide a protection to multiple internal organs contained within multiple cavities of the human body or a collection of anatomical structures. Examples of collections of anatomical structures include the abdomen, the thorax, the head, the neck or a limb. In some implementations, when the implantable body armor is intended to be implanted at a subcutaneous or a submuscular implantation site located on the head, the implantable body armor can be implanted, for instance, beneath the scalp or the epicranium. In such implementations, the implantation site of the implantable body armor relative to a location of the anatomical structures can be selected in accordance with a desired directional protection to be provided to the collection of anatomical structures. For instance, in some implementations, the implantation site of the implantable body armor can be superior, inferior, anterior, posterior, medial, lateral, proximal or distal to the collection of anatomical structures. In some implementations, the implantation site can be selected to provide an implantable body armor extending circumferentially around the collection of anatomical structures.

In some implementations, the implantation site can be the result of a creation of a space at a desired location. In other words, the implantation site may not be a physiological space, but can be created by the healthcare provider at a desired location where the implantable body armor is to be implanted. The creation of the space for the implantation site can be achieved for instance by displacing two physiological structures otherwise joined together and/or by surgically removing non-vital tissue occupying the implantation site.

In some implementations, the implantation of the implantable body armor at a given implantation site can contribute to reinforcing, supporting and/or repairing a hard tissue or a soft tissue located at this implantation site. For instance, the implantable body armor can be implanted at an implantation site having a wall defect, and the implantable body armor, in addition to the protection conferred by the flexible panel, can contribute to reinforce, support and/or repair the wall defect. Examples of wall defects include defects of the thoracic wall, suture line reinforcement, muscle flap reinforcement, hernia repair, soft tissue reconstructive procedures including plastic and reconstructive surgical applications, and for reinforcement of soft tissues which are repaired by suture or suture anchors.

In some implementations, an outer implantable body armor and an inner implantable body armor, together forming a pair of implantable body armors, can be implanted in the human body, with a physiological structure extending therebetween. For instance, the outer implantable body armor can be implanted at an implantation site located subcutaneously, on one side of the abdominal wall, and the inner implantable body armor can be implanted at an implantation site located on the other side of the abdominal wall, i.e., internally against the abdominal wall. In such implementations, the abdominal wall is thus sandwiched between the outer implantable body armor and the inner implantable body armor. A fixation mechanism, such as sutures, can then be used to engage the flexible panel with a soft tissue in the vicinity of the implantation sites, as will be described in further detail below. In some implementations, the fixation mechanism can be used to engage the outer implantable body armor and the inner implantable body armor through the abdominal wall (or through another physiological structure if the outer implantable body armor and the inner implantable body armor are implanted at other implantation sites).

The properties of the woven fibers can further enable providing a resulting implantable body armor suitable for implantation via an endoscopic procedure including, for instance, a third space endoscopy procedure, a videoscopic endoscopy procedure, a laparoscopic procedure, or a thoracoscopic procedure. In order to do so, the implantable body armor can be configurable between one of multiple configurations, including configurations that can contribute to facilitating a delivery of the implantable body armor to a desired implantation site. For instance, the implantable body armor can be configurable between a delivery configuration for delivery of the implantable body armor to the desired implantation site, and a functional configuration once the implantable body armor is positioned at the desired implantation site.

As used herein, the expression “impact-resistant” refers to a structure of the implantable body armor having properties suitable for absorbing the impact energy of a projectile directed to the impact-resistant structure and reducing or stopping the penetration of the projectile across a surface of the impact-resistant structure. In some implementations, the impact-resistant structure is configured, upon impact from a projectile, to spread the kinetic energy of the projectile over a surface area of the impact-resistant structure to halt its motion prior to further penetration into the implantable body armor, thereby preventing or limiting damage to the one or more internal organs being protected by the implantable body armor.

Various implementations and features of the implantable body armor for placement at an implantation site within a human body will now be described in greater detail in the following paragraphs.

### General Description of the Implantable Body Armor

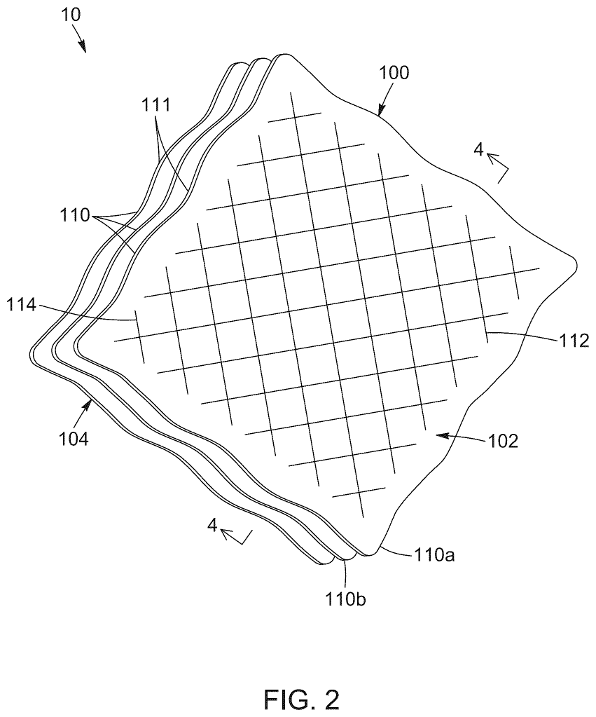

With reference to FIG. 2, an example of an implantable body armor 10 for placement at an implantation site within a cavity of a human body is shown. In the illustrated implementation, the implantable body armor 10 includes a flexible panel 100 configured to be impact-resistant to reduce the risk of injury to an internal organ within the cavity of the human body. The flexible panel 100 includes a cavity surface 102 configured to face the cavity once the implantable body armor 10 is implanted at the implantation site, and an outer surface 104 opposite the cavity surface 102. As will be discussed in further detail below, in some implementations, the implantable body armor 10 can be implanted within the thoracic cavity 3 with the outer surface 104 of the flexible panel 100 facing the thoracic cage, and for instance abutting or being secured to an interior surface of the thoracic cage. When the outer surface 104 is configured to face the thoracic cage, the outer surface 104 can be referred to as an “intercostal surface”.

The flexible panel 100 of the implantable body armor 10 can include layers of woven fibers 110 superposed to each other. In some implementations, the two outermost layers 111 of the superposed layers of woven fibers 110 can define the cavity surface 102 and the outer surface 104, respectively. In other implementations, the flexible panel 100 can include a panel cover at least partially covering the outermost layers 111 of the superposed layers of woven fibers 110, the panel cover comprising the cavity surface 102 and the outer surface 104 of the flexible panel 100. This aspect will be discussed in more detail below. In some implementations, the flexible panel 100 can include at least three superposed layers of woven fibers 110. In other implementations, the flexible panel 100 can include at least six superposed layers of woven fibers 110. In other implementations still, the flexible panel 100 can include at least ten superposed layers of woven fibers 110. It is to be understood that the flexible panel 100 can include any number of superposed layers of woven fibers that is sufficient to provide an impact-resistant structure while retaining a desired flexibility for implantation in the human body. The number of superposed layers of woven fibers 110 chosen to achieve these properties can depend on the material forming the woven fibers, for instance. In other words, depending on the type of woven fibers chosen, an implementation of the flexible panel 100 can include a higher number of superposed layers of woven fibers 110 to achieve an impact-resistant structure that remains flexible and suitable for implantation in the human body, compared to another type of woven fibers. In some implementations, a thickness of the flexible panel 100 can thus vary depending on the type of woven fibers chosen. In some implementations, a higher thickness of the flexible panel 100 can provide a higher degree of impact-resistance protection, while in other implementations, the degree of impact-resistance protection may not be related to the thickness of the flexible panel 100.

In some implementations, the layers of woven fibers 110 can be superposed in accordance with a layering pattern selected to improve the strength of the implantable body armor 10 and the ability to absorb and distribute the kinetic energy of a projectile. For instance, adjacent ones of the layers of woven fibers 110 can be superposed such that overall, the fibers of the layers of woven fibers 110 can be provided in a cross-layered arrangement in which the fibers 112 are superposed in alternating directions. In some implementations, the cross-layered arrangement can be achieved by having the fibers 112 of a first layer of woven fibers 110a run in a first direction (i.e., the fibers 112 forming the first layer of woven fibers 110a can run side-to-side between opposite edges of the first layer of woven fibers 110a), and the fibers 112 of a second layer of woven fibers 110b superposed to the first layer of woven fibers 110a can run in a second direction that is at an angle with respect to the first direction.

In some implementations, the layers of woven fibers 110 can be bound together using a resin or an adhesive to form a lamination of the flexible panel 100. In such implementations, the flexible panel 100 can be referred to as a laminated flexible panel. When the layers of woven fibers 110 are bound using a resin to achieve the lamination of the flexible panel 100, the layers of woven fibers 110 can be impregnated with the resin which can then be cured using heat and pressure. In some implementations, the resin can include a polyurethane-based resin, an epoxy resin, a thermoplastic resin, or a combination thereof. In some implementations, the implantable body armor 10 can include a fixation mechanism, as described in greater detail below, configured to engage the flexible panel 100 with a hard tissue and/or a soft tissue located in the vicinity of the implantation site. In such implementations, the fixation mechanism can additionally function to bind together one or more of the layers of woven fibers 110 when the implantable body armor 10 is implanted at the implantation site. When the implantable body armor 10 includes a fixation mechanism engaging each of the layers of woven fibers 100, the layers of woven fibers 100 can remain superposed relative to one another without requiring a resin or an adhesive.

In some implementations, the layers of woven fibers 110 can include fibers 112 suitable for forming an impact-resistant structure configured to absorb the kinetic impact energy of a projectile once woven together and combined as multiple layers. In some implementations, the fibers 112 of the layers of woven fibers 110 can be made of aramid fibers. In some implementations, the aramid fibers can include, for instance, para-aramid fibers. The para-aramid fibers can be poly-p-phenylene terephthalamide fibers, such as Kevlar™ fibers, Twaron™ fibers, or a combination thereof. In some implementations, the fibers 112 of the layers of woven fibers 110 can be made of polyethylene, such as ultra-high-molecular-weight polyethylene (UHMWPE). An example of UHMWPE fibers is Dyneema™ fibers.

In some implementations, the fibers 112 of the layers of woven fibers 110 can be bundled, for weaving, into a yarn 114 having at least 250 of the fibers 112. In other implementations, the yarn 114 can include at least 500 of the fibers 112. The yarn 114 can have a strength at break of at least 200 N, a tensile strength at break of at least 2.3 mN/tex and an elongation at break of between 2.2% and 3.8%. In some implementations, the fibers 112 of the layers of woven fibers 110 can have a fiber denier of 2 dtex or less, 1.1 dtex or less, or 0.55 dtex or less. In some implementations, the fibers 112 of the layers of woven fibers 110 can have a tensile strength of about 7 g/denier or more and/or a tensile modulus of about 150 g/denier or more. In some implementations, the fibers 112 of the layers of woven fibers 110 can have a density of less than 1.5 g/cm³.

As used herein, the term “woven” refers to an arrangement of the fibers 112 or the yarns 114 that are combined together to form the layers of woven fibers 110 according to a predetermined architecture. For instance, in the illustrated implementation, each of the layers of woven fibers 110 includes a plurality of yarns 114 of fibers 112 crossed over and under each other in an alternating pattern at intersection points 116, such that the woven pattern corresponds to a plain weave mesh pattern. It is to be understood that, in other implementations, the fibers 112 or yarns 114 can be woven in accordance with any other suitable woven pattern including, for instance, a twill weave, a plain dutch weave, a stranded weave, a twill dutch, an inter-crimp, or a lock crimp pattern. In other implementations still, the fibers 112 of the flexible panel 100 can be combined together in a substantially random fashion to form layers of fibers having no identifiable pattern.

In some implementations, adjacent ones of the fibers 112 or yarns 114 of the layers of woven fibers 110 can define interstices 118 into which cells of the biological tissue where the implantable body armor 10 is implanted can migrate and proliferate. The migration and proliferation of the cells of hard tissue, such as bone, and/or soft tissue can contribute to promoting growth of the hard tissue and/or soft tissue around or within the interstices 118 of the flexible panel 100 of the implantable body armor 10 to contribute to an integration of the flexible panel 100 into an adjoining tissue once the implantable body armor 10 is delivered at the implantation site. The distance between the adjacent ones of the fibers 112 or yarns 114 of the layers of woven fibers 110 and the woven pattern of the layers of woven fibers 110 define the sizing and the shape of the interstices 118, respectively. When an integration of the flexible panel 100 into an adjoining tissue is desired, at least one of the outermost layers 111 of the layers of woven fibers 110 can define interstices 118 sized to promote a growth of tissue therebetween. In some implementations, one or more of the layers of woven fibers 110 positioned between the outermost layers 111 can further include interstices 118 sized to promote a growth of tissue therebetween. When the interstices 118 of the layers of woven fibers 110 are positioned consecutively beyond the outermost layers 111 overlap at least partially, cells of the biological tissue where the implantable body armor 10 is implanted can migrate and proliferate through multiple ones of the layers of woven fibers 110 to provide a further integration of the flexible panel 100 into the adjoining tissue compared to an integration of adjoining tissue occurring at the level of the outermost layers 111 only.

In some implementations, the distance between the adjacent ones of the fibers 112 or yarns 114 of the layers of woven fibers 110 can be reduced to reduce the size of the interstices 118 and provide a layer of woven fibers 110 with a higher density, if desired. In some implementations, a layer of woven fibers 110 with a higher density can contribute to increasing the high energy absorption characteristics of the flexible panel 100 once multiple layers of woven fibers 110 are combined together. It is to be understood that in some implementations, the flexible panel 100 can include a first set of layers of woven fibers 110 having given characteristics and a second set of layers of woven fibers 110 having other given characteristics that may be different than those of the first set of layers of woven fibers, in order to provide overall characteristics to the flexible panel 100 that are sought after.

In some implementations, a given one of the outermost layers 111 can include interstice zones provided at selected location(s) on the given one of the outermost layers 111, each of the interstice zones covering a portion of a surface area of the given one of the outermost layers 111 and including interstices 118 having a greater or smaller size relative to a size of the interstices 118 provided along the remaining surface area of the given one of the outermost layers 111. A variation in the size of the interstices 118 at these selected location(s) along the given one of the outermost layers 111 may provide a different degree of integration of the flexible panel 100 into the adjoining tissue compared to the remaining surface area of the given one of the outermost layers 111. For instance, the given one of the outermost layers 111 can define a first interstice zone in which the fibers 112 or yarns 114 define first zone interstices 118 having a first size selected to promote a growth of tissue therebetween (i.e., into which cells of the biological tissue where the implantable body armor 10 is implanted can migrate and proliferate), and a second interstice zone in which the fibers 112 or yarns 114 can define second zone interstices 118 having a second size that is smaller than the first size of the first zone interstices 118, the second size being selected to as to be sufficiently small to reduce or prevent migration and proliferation of cells of the biological tissue therein, thus remaining substantially unadhered from the adjoining tissue along the second interstice zone of the given one of the outermost layers 111. In some implementations, when an unadherence of the flexible panel 100 along at least a portion of a periphery thereof is desirable, the first interstice zone can extend over a central region of the give one of the outermost layers 111, and the second interstice zone can extend over a peripheral region of the given one of the outermost layers 111, i.e., the second interstice zone can surround the first interstice zone, thus extending around the central region. In such implementations, the implantable body armor 10 can provide an increased flexibility along the periphery of the flexible panel 100. It is to be understood that the outermost layers 111 of the layers of woven fibers 110 can include any number of interstice zones having interstices 118 of varying sizes to provide a desired degree of integration at given locations on the given one of the outermost layers 111 of the flexible panel 100, and that the configuration described above (central region and peripheral region) is presented for exemplary purposes only.

In some implementations, the flexible panel 100 can include a coating (e.g., a resin or any other biocompatible coating), such as a friction-reducing coating, provided onto the second interstice zone of the given one of the outermost layers 111 to reduce a size of the interstices 118 relative to the size of the interstices of the first interstice zone, thus reducing or preventing migration and proliferation of cells of the biological tissue in the interstices 118 of the second interstice zone. In such implementations, a degree of integration of the flexible panel 100 into the adjoining tissue once the implantable body armor 10 is delivered at the implantation site can vary depending on the location of the second interstice zone along the given one of the outermost layers 111, irrespective of the size of the interstices 108 defined between the adjacent ones of the fibers 112 or yarns 114 provided in the second interstice zone. In such implementations, the flexible panel 100 can adhere to the adjoining tissue along the first interstice zone of the given one of the outermost layers 111, while remaining substantially unadhered from the adjoining tissue along the second interstice zone of the given one of the outermost layers 111 to provide a greater range of motion along the second interstice zone with respect to the organ(s) the given one of the outermost layers 111 is facing. The substantial unadherence of the flexible panel 100 along a portion thereof may be desirable when, for instance, the implantable body armor 10 is implanted in the vicinity of an internal organ undergoing periodic motion, such as the heart, the lung or a muscle as they undergo periodic contraction and expansion without interfering with the proper physiological functioning of the internal organ or the group of internal organs.

In some implementations, one or more of the interstice zones can include discontinued regions together forming the resulting interstice zone. In other words, the interstice zone can include discontinued regions of the surface area of the given one of the outermost layers 111 that do not intersect or share any common points. For instance, in some implementations, the first interstice zone can include two discontinued regions provided laterally opposite to each other on the surface area of the given one of the outermost layers 111, with no overlap between the two discontinued regions.

In some implementations, given layers of woven fibers 110 of the flexible panel 100 can include fibers from different sources. For instance, in some implementations, a first set of layers of woven fibers 110 forming the flexible panel 100 can be made of aramid fibers, and a second set of layers of woven fibers 110 can be made of UHMWPE fibers.

In some implementations, a given layer of woven fibers 110 can also include fibers from different sources. For instance, in some implementations, the given layer of woven fibers 110 can include at least two types of fibers. In some implementations, the given layer of woven fibers 110 can include for instance aramid fibers and UHMWPE fibers.

In some implementations, the given layer of woven fibers 110 can include for instance aramid fibers and/or UHMWPE fibers, and additional fibers that are different from the aramid fibers and/or UHMWPE fibers. The additional fibers can be selected to provide additional beneficial properties to the flexible panel 100. For instance, in some implementations, the layers of woven fibers 110 can include additional fibers made of natural or synthetic fibers having hydrophobic properties, which can facilitate repelling or inhibiting fluid penetration into the interstices 118 defined between adjacent fibers 112 or adjacent yarns 114 of the layers of woven fibers 110. In some implementations, the hydrophobic properties of the additional fibers can further prevent bacterial adhesion thereto, which in turn can contribute to preventing an infection at the implantation site.

In some implementations, the additional fibers of the layers of woven fibers 110 can be bioabsorbable and thus capable of being absorbed within a period of time at physiological conditions. The absorbable nature of the additional fibers of the layers of woven fibers 110 can stem from their susceptibility to breakdown, decomposition, degradation, resorption and/or dissolution, under the action of biological processes, such as enzymatic reaction and/or hydrolysis. In such implementations, the additional fibers of the layers of woven fibers 110 can be progressively absorbed during a given period of time, such that the subsequent absence of these additional fibers following absorption can contribute to define a greater distance between adjacent ones of the remaining fibers 112 and thus larger interstices 118. Thus, in a first phase following implantation of the implantable body armor 10 at the implantable site, the additional fibers can be absorbed during a given period of time, and in a second phase, following or overlapping the first phase, the larger interstices 118 can contribute to promote growth of hard tissue and/or soft tissue within the interstices defined in the layers of woven fibers 110 of the flexible panel 100. As mentioned above, the growth of hard tissue and/or soft tissue within the interstices 118 can contribute to implant the flexible panel 100 into an adjoining tissue at the implantation site within the cavity of the human body after a predetermined period of time.

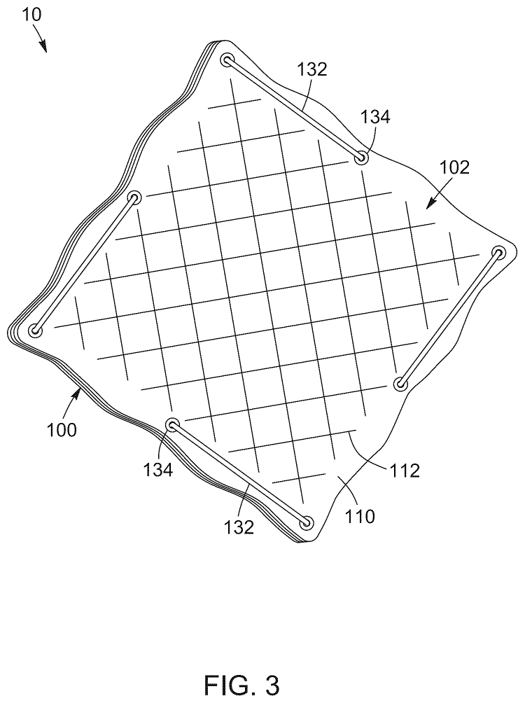

In some implementations, the implantable body armor 10 can include additional features that can contribute to securing the implantable body armor 10 to an internal organ, a soft tissue and/or a hard tissue such as bone once positioned at the implantation site. Referring to FIG. 3, an example of the implantable body armor 10 is shown including a fixation mechanism 132 configured to engage the flexible panel 100 with an internal organ, a bone and/or a soft tissue located in the vicinity of the implantation site. In some implementations, the fixation mechanism 132 can be selected in accordance with the characteristics of the internal organ, the bone and/or the soft tissue located in the vicinity of the implantation site. For instance, in some implementations, the fixation mechanism 132 can be configured to engage the flexible panel 100 with a soft tissue in the vicinity of the implantation site. For instance, in the implementation shown, the fixation mechanism 132 is a surgical suture configured to engage the flexible panel 100 with a soft tissue in the vicinity of the implantation site. In some implementations, the fixation mechanism 132 can include for instance surgical staples, other forms of surgical sutures (such as piercing sutures), a biological adhesive, a cyanoacrylate adhesive and internal buttons. In some implementations, the fixation mechanism 132 can be configured to engage the flexible panel 100 with a hard tissue in the vicinity of the implantation site and can include, for instance, interference screws, transfixion pins, bone tacks, spinal tacks, rivets, screws and suture anchors. In some implementations, the fixation mechanism 132 configured to engage the flexible panel 100 with a hard tissue can be made of medical grade metal, such as titanium.

In some implementations, the flexible panel 100 can include a fixation mechanism engaging feature 132 provided along an outer periphery 106 of the flexible panel 100. The fixation mechanism engaging feature 134 is configured to be engaged by the fixation mechanism 132 for the securing of the flexible panel 100 to the internal organ, hard tissue and/or soft tissue. For instance, as exemplified in the implementation shown, the fixation mechanism engaging feature 134 can include an attachment aperture sized to receive the fixation mechanism 132. In other implementations, the fixation mechanism engaging feature 134 can define a portion of the outer periphery 106 of the flexible panel 100 being penetrable by a portion of the fixation mechanism to secure the flexible panel 100 to the hard tissue and/or soft tissue.

In some implementations, one or both of the fixation mechanism 132 and the fixation mechanism engaging feature 134 can be made of a fixation mechanism material having properties which can vary to modify one or more characteristics thereof within a period of time following an implantation of the implantable body armor 10 at the implantation site. For instance, in some implementations, the fixation mechanism can be bioabsorbable, and one or both of the fixation mechanism 132 and the fixation mechanism engaging feature 134 can be absorbed within a period of time corresponding to a period of time sufficiently long to enable an integration of at least one of the cavity surface 102 and the outer surface 104 of the flexible panel 100 into an adjoining tissue following the delivery of the implantable body armor 10 at the implantation site, as described above. Accordingly, when an unadherence of the flexible panel 100 along at least a portion of a periphery thereof is desirable, the implantable body armor 10 can return to a state where mobility of the periphery of the flexible panel 100 relative to adjoining tissue is recovered following an absorption of the fixation mechanism 132 and/or the fixation mechanism engaging feature 134, when the periphery of the flexible panel 100 is substantially unadhered to adjoining tissue. The absorbable nature of the fixation mechanism 132 and/or the fixation mechanism engaging feature 134 can stem from its susceptibility to breakdown, decomposition, degradation, resorption and/or dissolution, under the action of biological processes, such as enzymatic reaction and/or hydrolysis. In some implementations, the fixation mechanism 132 and/or the fixation mechanism engaging feature 134 can be made of an aliphatic polyester or a combination of aliphatic polyesters, or can include one or more portions made of an aliphatic polyester or a combination of aliphatic polyesters. The aliphatic polyester can be a synthetic aliphatic polyester. Examples of aliphatic polyester include polylactic acid, polyglycolic acid, polylactic-co-glycolic acid, polycaprolactone, and polydioxanone.

The flexible panel 100 of the implantable body armor 10 can have any suitable shape and size that can be determined in accordance with various factors. For instance, in some implementations, the size and the shape of the flexible panel 100 can be determined in accordance with the shape and size of the internal organ or the group of internal organs for which protection by the implantable body armor 10 is desired. In some implementations, the size and the shape of the flexible panel 100 can be determined based on the characteristics of the implantation site where the implantable body armor 10 is intended to be implanted. In some implementations, the size and the shape of the flexible panel 100 can be determined in accordance with the delivery technique chosen to deliver the implantable body armor 10 to the implantation site. It is to be understood that the above factors are given as examples only and should not be interpreted in a restrictive sense.

In some implementations, the flexible panel 100 can have for example an elliptic shape, a circular shape, an elongated shape, a rectangular shape, an octagonal shape, or any other polygonal shape. In some implementations, when the implantable body armor 10 has a polygonal shape, the implantable body armor 10 can have rounded corners to prevent an undesired irritation or damage to surrounding tissue during delivery of the implantable body armor 10 to the implantable site.

In some implementations, the flexible panel 100 can be substantially flat. It is to be understood that when referring to the flexible panel 100 being substantially flat, it is intended to describe the flexible panel 100 when not subjected to any constraint or prior to the flexible panel 100 being given a desired shape. The flexible panel 100 can then be given any suitable shape, for instance in accordance with the various factors mentioned above or others. Accordingly, the flexible panel 100, similarly to a piece of fabric, can be given a curved shape to mate with a curved surface of an internal organ or a curved surface of a peripheral wall of the cavity of the human body, for instance. In some implementations, the flexible panel 100 can remain substantially flat once implanted at the implantation site, for instance if the intended surface to be protected is large and the implantable body armor 10 is implantable adjacent to the thoracic cage, inwardly or outwardly thereof. In such implementations, the flexible panel 100 can be given a slight curvature corresponding to the curvature of the thoracic cage, although this curvature will have a smaller amplitude than a curvature resulting from the flexible panel 100 being placed to mate with an internal organ.

In some implementations, at least one of the outermost layers 111 of the layers of woven fibers 110 forming the flexible panel 100 can include a biocompatible coating. The biocompatible coating can contribute to facilitate integration of the implantable body armor 10 at the implant site and reduce risks of a potential adverse reaction occurring at the implantation site due to a poor interaction between the implantable body armor 10 and surrounding tissues. In some implementations, when there may be a risk that the type of fibers chosen to produce the layers of woven fibers 110 may have a potential of rejection by the body once the implantable body armor 10 would be implanted at the implantation site, the biocompatible coating can advantageously reduce the risk of such rejection by avoiding a direct contact of the fibers with biological fluids, soft tissues, internal organs, etc.

In some implementations, the biocompatible coating can be deposited onto the fibers 112 prior to the production of a layer of woven fibers 110. Thus, instead of applying the biocompatible coating onto the cavity surface 102 and/or the outer surface 104 once the layers of woven fibers 110 are produced, the biocompatible coating can be applied to the fibers intended to be used to produce the outermost layers 111 of the layers of woven fibers 110 providing the cavity surface 102 and the outer surface 104. Examples of suitable biocompatible coatings include hydroxyapatite coatings, cross-linked polyethylene, polylactic acid (PLA), polylactic-co-glycolic acid (PLGA), silicone elastomers, polyurethane (PU) coatings, polycaprolactone (PCL), chitosan coatings, graphene oxide coatings, collagen, among others.

In some implementations, the biocompatible coating can contribute to providing a selected one of, or both, the cavity surface 102 and the outer surface 104 that is smoother, i.e., with a reduced coefficient of friction, compared to when no biocompatible coating is present. Providing the selected one of, or both, the cavity surface 102 and the outer surface 104 that is smoother compared to when no biocompatible coating is present can be beneficial for instance for reducing the friction generated by the flexible panel 100 relative to a surface of an internal organ that it is intended to protect and/or relative to the thoracic cage once delivered to the implantation site. In other implementations, the biocompatible coating can be given characteristics that can contribute to facilitate implantation of the flexible panel 100 at the implantation site. For instance, the biocompatible coating can contribute to provide a surface roughness to a selected one of, or both, the cavity surface 102 and the outer surface 104. The surface roughness can have a similar effect as the interstices 118 between adjacent fibers of the layer of woven fibers 110 by promoting growth of tissues within recesses of the surface roughness. For instance, the surface roughness of a given one of the cavity surface 102 and the outer surface 104, or of both the cavity surface 102 and the outer surface 104, can contribute to facilitate collagen deposition onto the corresponding surface. In some implementations, one of the cavity surface 102 and the outer surface 104 can have a biocompatible coating that results in the surface being smooth, and the other one of the cavity surface 102 and the outer surface 104 can have a compatible coating that results in the surface having a surface roughness. For instance, it may be desirable to provide an outer surface 104 that is smooth, and to provide a cavity surface 102 that has a surface roughness, or vice versa, depending on the location and characteristics of the implantation site. For instance, in some implementations, the cavity surface 102 can have a cavity surface roughness that is higher than an intercostal surface roughness of the outer surface 104. In some implementations, the biocompatible coating of the flexible panel 100 can further be configured to prevent or reduce deleterious modifications to the layers of woven fibers 110 of the flexible panel 100 under physiological conditions. It is to be understood that the term “coated” is not intended to limit the method by which the biocompatible coating is applied onto the flexible panel 100. In addition, the description made above regarding the surface roughness of the cavity surface 102 and the outer surface 104 and the smoothness of the cavity surface 102 and the outer surface 104 can also be applicable directly to the flexible panel 100 when no biocompatible coating is provided, for instance by providing a surface treatment to the cavity surface 102 and/or the outer surface 104.

In some implementations, the biocompatible coating can include a bioactive agent selected so as to induce or facilitate a desired biological response. In some implementations, the bioactive agent can be selected to facilitate integration of the flexible panel 100 at the implantation site. In some implementations, the bioactive agent can include a therapeutic agent. The bioactive agent can include, for instance, a cell growth promoter, a cell growth inhibitor, an antibiotic, a cytokine, a healing promoter, a clotting modulator, an anti-inflammatory, and an anti-scarring agent. In some implementations, the bioactive agent can include an osteoconductive bone adhesive, calcium carbonate, fatty acids, a lubricant, and/or an antiseptic. In some implementations, the bioactive agent can be combined with the fibers 112 of the layers of woven fibers 110 other than via a biocompatible coating.

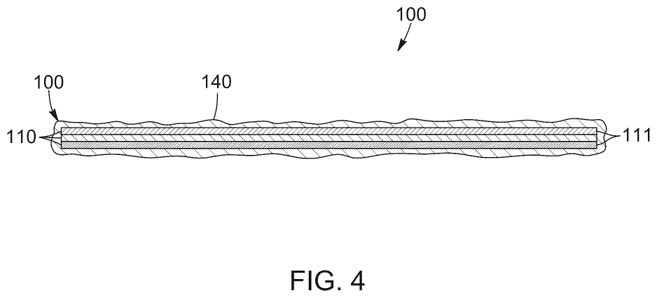

Referring now to FIG. 4, in some implementations, the implantable body armor 10 can further include a panel cover 140 at least partially covering the flexible panel 100. The panel cover 140 can be made of a biocompatible material. The panel cover 140 can contribute to facilitate integration of the implantable body armor 10 at the implant site and reduce risks of a potential adverse reaction occurring at the implantation site due to a poor interaction between the implantable body armor 10 and surrounding tissues.

In some implementations, the panel cover 140 can contribute to providing a selected one of, or both, the cavity surface 102 and the outer surface 104 that is smoother, i.e., with a reduced coefficient of friction, compared to when no panel cover 140 is present. Providing the selected one of, or both, the cavity surface 102 and the outer surface 104 that is smoother compared to when no panel cover 140 is present can be beneficial for instance for reducing the friction generated by the flexible panel 100 relative to another surface such as a surface of an internal organ that it is intended to protect and/or relative to the thoracic cage once delivered to the implantation site. In other implementations, the panel cover 140 can be given characteristics that can contribute to facilitate implantation of the flexible panel 100 at the implantation site. For instance, the panel cover 140 can contribute to provide a surface roughness to a selected one of, or both, the cavity surface 102 and the outer surface 104. The surface roughness can have a similar effect as the interstices 118 between adjacent fibers 112 of the layer of woven fibers 110 by promoting growth of tissue within recesses of the surface roughness. In some implementations, the panel cover 140 can result in one of the cavity surface 102 and the outer surface 104 being smooth, and the other one of the cavity surface 102 and the outer surface 104 having a surface roughness. For instance, it may be desirable to provide an outer surface 104 that is smooth, and to provide a cavity surface 102 that has a surface roughness, or vice versa, depending on the location and characteristics of the implantation site. In some implementations, the panel cover 140 of the flexible panel 100 can further be configured to prevent or reduce deleterious modifications to the layers of woven fibers 110 of the flexible panel 100 under physiological conditions.

In some implementations, the panel cover 140 can include a bioactive agent selected so as to induce or facilitate a desired biological response. Examples of bioactive agents are given above.

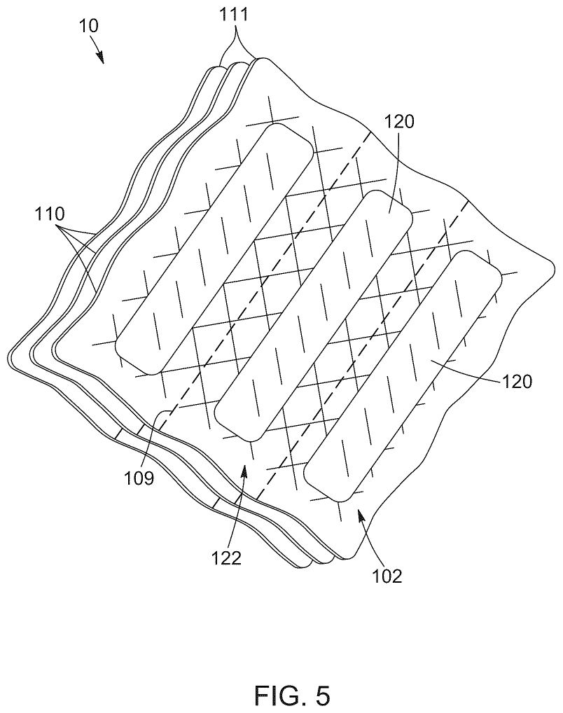

Referring to FIG. 5, in some implementations, the implantable body armor 10 can further include one or more reinforcement zones 120 provided at a selected location or in selected locations, respectively. The selected location(s) can be chosen such that once the implantable body armor 10 is implanted at the implantation site, the reinforcement zone(s) 120 is (are) located at sensitive site(s) where enhanced protection may be desired. Examples of sensitive sites can include vital organs such as the heart and the lungs, among other examples. The reinforcement zones 120 are configured to provide an additional impact energy absorption to the selected locations. In the illustrated implementation, the reinforcement zones 120 are spaced apart and extend in selected regions of the flexible panel 100 to provide an additional impact energy absorption compared to one or more unreinforced zones 122 of the flexible panel 100, i.e., zones of the flexible panel 100 where no reinforcement is present. In some implementations, the reinforcement zones 120 can include a higher number of layers of woven fibers 110 extending over the selected regions of the flexible panel 100. Alternatively, in other implementations, the reinforcement zones 120 can include metal armor or ceramic armor secured to one of the cavity surface 102 and the outer surface 104 of the flexible panel 100. Alternatively, the reinforcement zones 120 can be provided between adjacent ones of the superposed layers of woven fibers 110 of the flexible panel 100, i.e., sandwiched between two adjacent ones of the superposed layers of woven fibers 110. Examples of suitable metal armor or ceramic armor include MIL-A-46100 steel, AR500 steel, titanium, aluminum, boron carbide, silicon carbide, alumina, among others. In some implementations, the unreinforced zones 122 of the flexible panel 100 between adjacent ones of the reinforcement zones 120 can be sized and configured to retain a desirable flexibility of the implantable body armor 10.

### Delivery of the Implantable Body Armor

As stated above, in some implementations, the implantable body armor 10 can be configurable between a delivery configuration and a functional configuration. The two configurations can contribute to facilitating the delivery of the implantable body armor 10 at the implantation site and the subsequent implantation of the implantable body armor 10 at the implantation site. Accordingly, the implantable body armor 10 can be configured in a delivery configuration during delivery of the implantable body armor 10 to the implantation site, and a functional configuration once the implantable body armor 10 is positioned at the desired implantation site.

In such implementations, the implantable body armor 10 can be configurable between a delivery configuration in which a dimension of the flexible panel 100 is decreased and/or a shape of the flexible panel 100 is modified to facilitate a delivery of the implantable body armor 10 to the implantation site via a delivery path extending between the outside of the human body, i.e., at the skin level, to the implantation site. The dimension and/or the shape of the flexible panel 100 can be modified to facilitate a passage of the implantable body armor 10 through a channel or opening of the human body defining the delivery path and leading to the implantation site. In some implementations, the delivery path can be created by performing a percutaneous incision at a selected location. For instance, when the implantation site is located within one of the thoracic cavity 3 or the dorsal cavity 7, the percutaneous incision can extend through an intercostal space and into the corresponding one of the thoracic cavity 3 and the dorsal cavity 7. Other delivery paths can of course be used depending on the choice of implantation site. In addition, the delivery configuration of the of the implantable body armor 10 can enable a health care provider in charge of the implantation of the implantable body armor 10 to use a minimally invasive approach by passing the flexible panel 100 through a smaller percutaneous incision than if the implantable body armor 10 was not configurable in a delivery configuration, while reducing potential trauma that could occur to surrounding tissues during delivery of the implantable body armor 10 to the implantation site.

In some implementations, the reduced dimension and/or the modified shape of the flexible panel 100 can be achieved by rolling the flexible panel 100 onto itself or folded onto itself, such that the surface area of the resulting external surface remaining after the rolling or the folding, i.e., the surface of the flexible panel 100 exposed to the surrounding environment, external to the implantable body armor 10, is decreased compared to when the flexible panel 100 is not rolled or folded onto itself. Reducing the surface area of the resulting external surface of the flexible panel 100 can facilitate the delivery of the implantable body armor 10 to the implantation site, because an object having a smaller surface area can be easier to navigate through the delivery path without inducing potential trauma to surrounding tissues.

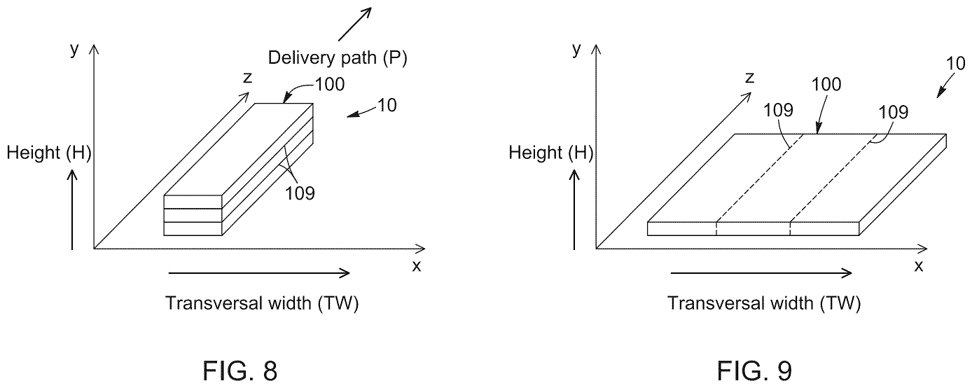

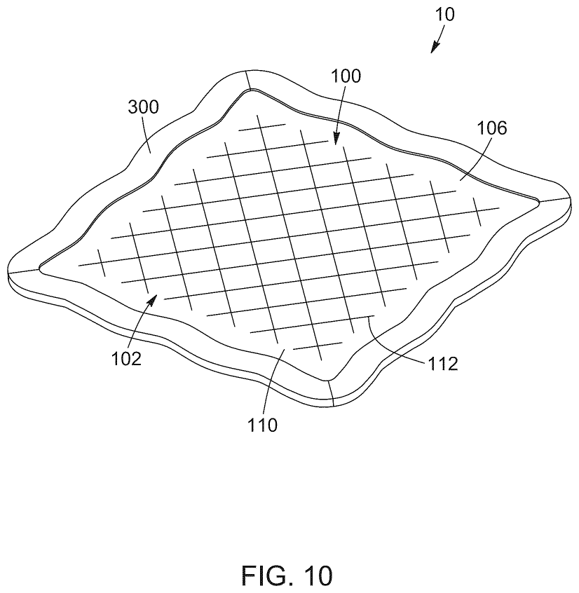

In some implementations, the reduced dimension and/or the modified shape of the flexible panel 100 can be achieved by rolling the flexible panel 100 onto itself or by folding the flexible panel 100 onto itself, such that the cross-section of the flexible panel 100 is altered compared to when the flexible panel 100 is not rolled or folded onto itself, i.e., in its functional configuration. The cross-section of the flexible panel 100 is characterized by an overall height and transversal width of the flexible panel 100. With reference to FIG. 8, when the flexible panel 100 is in the delivery configuration, the height and the transversal width of the flexible panel 100 can be defined as the greatest vertical extent (measured along the y axis) and the greatest horizontal extent (measured along the x axis) of the flexible panel 100, respectively, the x-y plan formed by the x and y axis corresponding to a transverse plane extending transversally with respect to an intended delivery path P of the implantable body armor 10 along axis z. Referring now to FIG. 9, when the flexible panel 100 is in the functional configuration, the height of the flexible panel 100 can be measured, using the same transversal plane as when the flexible panel 100 is in the delivery configuration, this time as a distance between the cavity surface 102 and the outer surface 104 still measured along the y axis, and the transversal width of the flexible panel 100 still measured along the x axis as a distance between opposing transversal edges of the flexible panel 100 (i.e., a distance between furthermost points of the flexible panel 100 along the x axis). The flexible panel 100 can thus include a first aspect ratio corresponding to a ratio between a transversal width and a height (referred to below as “aspect ratio TW:H”) when the implantable body armor 10 is in the functional configuration to provide a first cross-section of the implantable body armor 10 determined in accordance with the shape and size of the internal organ or the group of internal organs for which protection by the implantable body armor 10 is desired, and a second aspect ratio when the implantable body armor 10 is in the delivery configuration to provide a second cross-section of the implantable body armor 10 determined in accordance with the cross-sectional area measured in the x-y plan of the desired delivery path P extending to the implantation site. In some implementations, the flexible panel 100 can have a first aspect ratio TW:H (i.e., when the flexible panel 100 is in the functional configuration) of about 5:1, or a first aspect ratio of about 4:1 to about 20:1, about 10:1 to about 30:1, about 15:1 to about 50:1, or another aspect ratio. In some implementations, the second aspect ratio TW:H (i.e., when the flexible panel 100 is in the delivery configuration) can be smaller than the first aspect ratio. For instance, the flexible panel 100 can have a second aspect ratio of about 1:1 (e.g., when the flexible panel 100 is rolled onto itself), or a second aspect ratio of about 0.75:1 to about 1.5:1, about 0.5:1 to about 2:1, about 1.5:1 to about 5:1, about 1.5:1 to about 4:1, or another aspect ratio.

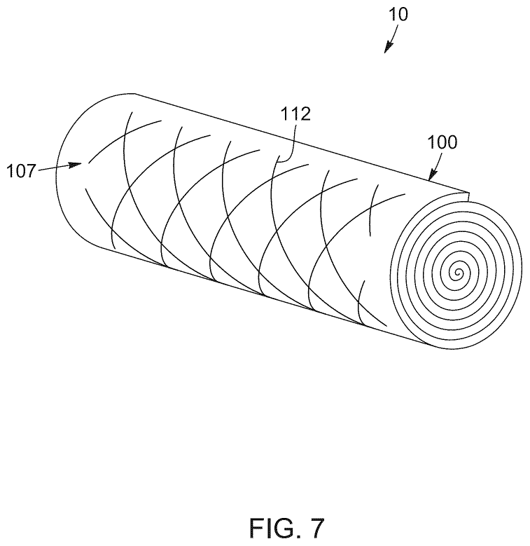

FIG. 7 illustrates an implementation of the implantable body armor 10 shown in a delivery configuration. In this implementation, the flexible panel 100 of the implantable body armor 10 is rolled-up onto itself to form a flexible panel roll having a cylindrical shape. In this configuration, given that the flexible panel 100 is rolled onto itself, the resulting external surface 107 of the implantable body armor 10 is reduced relative to the surface area of the cavity surface 102 or the outer surface 104. In the illustrated implementation, the flexible panel 100 is shown rolled-up such that the cavity surface 102 is curved toward and atop the outer surface 104 to overlap the outer surface 104, or vice versa, thereby reducing the surface area of the resulting external surface 107.

In some implementations, when one of the cavity surface 102 and the outer surface 104 of the flexible panel 100 is smoother than the other as described above, the flexible panel 100 can be configured such that the smoother of the cavity and outer surfaces 102, 104 forms the resulting external surface 107 of the implantable body armor 100 when the flexible panel 100 is in the delivery configuration, such that the smoother resulting external surface 107 can facilitate an implantation of the implantable body armor 10 to the implantation site.

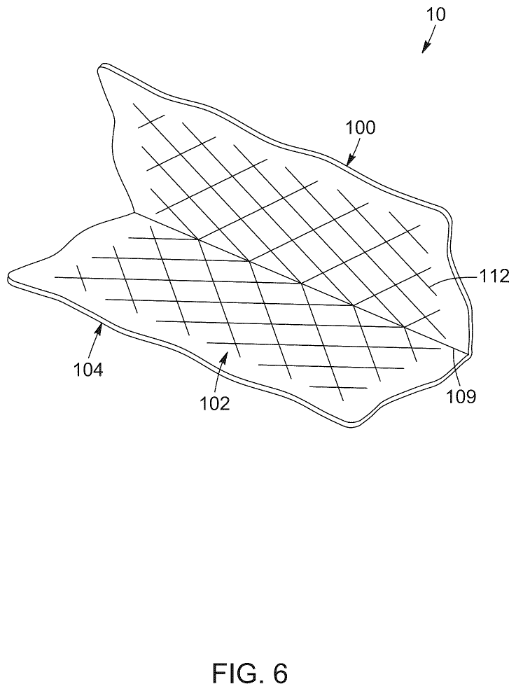

Referring to FIGS. 5 and 6, in order to fold the flexible panel 100 onto itself to reduce the surface area of the resulting external surface 107 remaining after the folding and thus place the implantable body armor 10 in the delivery configuration, the flexible panel 100 can include one or more folding lines 109 to facilitate such folding. In some implementations, the folding line(s) 109 can be provided at a predetermined location, or at predetermined locations if more than one is present, to facilitate achieving a delivery configuration that is predictable and suitable for delivery through a chosen delivery path of the flexible panel 100 to facilitate an implantation of the implantable body armor 10. For instance, when the implantable body armor 10 includes the reinforcement zones 120 described above, the folding lines 109 can be provided along one or more of the unreinforced zones 122 of the implantable body armor 10. In some implementations, the reinforcement zone 120 comprises first and second reinforcement zones 122 located in first and second selected regions, respectively, of the layers of woven fibers 110, the folding line 109 extending between the first and second reinforcement zones, and thus within an unreinforced zone 122, as shown for instance in FIG. 5. In FIG. 5, there are three reinforcement zones 122, with a first folding line 109 provided in between the first and second reinforcement zones 122 and a second folding line 109 provided in between the second and third reinforcement zones 122. In some implementations, the flexible panel 100 comprises one or more folding lines 109 extending at corresponding predetermined locations to facilitate a transition of the implantable body armor 10 between the delivery configuration and the functional configuration. FIG. 6 illustrates the implantable body armor 10 in a transitional state between the functional configuration and the delivery configuration with the flexible panel 100 being partially folded onto itself along a folding line 109. Although the flexible panel 100 illustrated in FIG. 6 is shown without reinforcement zones 120, it should be understood that the flexible panel 100 of FIG. 6 can alternatively be similar to the flexible panel 100 shown in FIG. 5 and can thus include the reinforcement zones 120 shown in FIG. 5.

In some implementations, a flexibility of the flexible panel 100 can vary along one or more of given axes of the flexible panel 100 selected in accordance with various factors. For instance, a flexibility of the flexible panel 100 along a longitudinal axis of the flexible panel 100 can differ from a flexibility of the flexible panel 100 along a transverse axis of the flexible panel 100. In some implementations, the flexibility of the flexible panel 100 can vary in accordance with a weave of the fibers 112 or yarns 114 of the layers of woven fibers 110 with the weave being denser in one direction (typically the warp or lengthwise direction) to improve resistance to impacts and penetration, while being more flexible in the other direction (the weft or crosswise direction). In some implementations, the flexibility of the flexible panel 100 can further vary in accordance with the layering technique of the layers of woven fibers 110, a coating of the flexible panel 100 and/or the presence of a reinforcement zone 120, as described above. When the flexibility of the flexible panel 100 varies along different directions, the implantable body armor 10 can be configured to fold or roll onto itself along a direction of greater flexibility. For instance, when a flexibility of the flexible panel 100 is greater along the weft direction of the weave of the fibers 112 or yarns 114 of the layers of woven fibers 110 relative to a warp direction thereof, the folding lines can be provided along the weft direction of the weave of the fibers 112 or yarns 114 of the layers of woven fibers 110 to facilitate a transition of the implantable body armor 10 between the delivery and functional configurations.

In some implementations, when in the delivery configuration, the implantable body armor 10 including the features described above can be navigated to the implantation site within the cavity of the human body, laparoscopically or thoracospically. In such implementations, the implantable body armor 10 can be configured for coupling with a delivery system. The coupling of the implantable body armor 10 and the delivery system can be achieved via any type of engagement considered suitable for successfully navigating the implantable body armor 10 through the delivery path while the implantable body armor 10 is in the delivery configuration. It is to be understood that although reference above is made to a delivery configuration and a functional configuration as two distinct configurations, in other implementations, the delivery configuration and the functional configurations can be substantially the same other than with respect to the immediate environment of the implantable body armor 10, i.e., at the implantation site or not. Examples of suitable delivery systems include a laparoscopic tool, a thoracoscopic tool, or any other suitable surgical tool suitable for navigating the implantable body armor 10 to the implantation site.

In some implementations, the delivery system can be configured to selectively deploy the implantable body armor 10 to the functional configuration once delivered to the implantation site. In some implementations, the delivery system can be further configured to position the implantable body armor 10, once in the functional configuration, in a position suitable for the flexible panel 100 to be secured to, or engaged with, an internal organ, a bone and/or a soft tissue located in the vicinity of the implantation site.

In some implementations, the implantable body armor 10 can further include an attachment feature configured to be releasably engageable with a connector of the delivery system. More specifically, the attachment feature can enable a connection between the implantable body armor 10 and a connector of the delivery system for navigating the implantable body armor 10 to the implantation site. The attachment feature can include any feature that enables a releasable connection of the implantable body armor 10 with the connector of the delivery system. For instance, in some implementations, the attachment feature can be a “catheter attachment” configured as a receiving cavity that can receive a distal end of the connector therein. In other implementations, the attachment feature can include the fixation mechanism engaging feature 134 of the flexible panel 100 described above. In such implementations, the fixation mechanism engaging feature 134 of the flexible panel 100 can remain accessible when the implantable body armor 10 is in the delivery configuration to receive a distal end of the connector therein. In some implementations, the connector of the delivery system can be releasably connectable to the attachment feature of the implantable body armor 10 through an interference fit and configured to release the implantable body armor 10 from the connector of the delivery system when the delivery system is subjected to a suitable pulling force or another disengagement maneuver, such as a rotation of the catheter attachment within the receiving cavity. When the disengagement maneuver includes a pulling force, the pulling force suitable for releasing the connector of the delivery system from the attachment feature of the implantable body armor 10 can be sufficient to overcome the engagement of the connector of the delivery system with the attachment feature of the implantable body armor 10, while being insufficient to undesirably displace the implantable body armor 10, now in the functional configuration, from the desired implantation site when the delivery system is pulled back, i.e., retrieved from the human body.

### Implantable Body Armor With an Implant Frame

Referring to FIG. 8, in some implementations, the implantable body armor 10 furthers include an implant frame 300. In the illustrated implementation, the implant frame 300 extends around the outer periphery 106 of the flexible panel 100 and is engaged with the flexible panel 100 so that the combination of the implant frame 300 and the flexible panel 100 can form a unitary structure, at least when the implantable body armor 10 is in the delivery configuration. In other implementations, the implant frame 300 can extend around a portion of the outer periphery 106 of the flexible frame 100 or engage any other suitable portion of the flexible panel 100 including, for instance, one or more of the cavity and outer surfaces 102, 104.

In some implementations, the implant frame 300 is configured to transition the implantable body armor 10 from a delivery configuration to a functional configuration. In such implementations, the implant frame 300 can be configurable between an undeployed configuration to place the implantable body armor 10 in the delivery configuration, and a deployed configuration to place the implantable body armor 10 in the functional configuration once at the implantation site. In some implementations, the deployed configuration of the implant frame 300 can be selected such that once the implantable body armor 10 is delivered to the implantation site, the flexible panel 100 is extended to cover the one or more internal organs for which the implantable body armor 10 is intended to provide protection, or to extend portions of the flexible panel 100 to pre-determined locations in the cavity of the human body where the flexible panel 100 is intended to be engaged with or be secured to.

In some implementations, the implant frame 300 can be configured as a multi-component frame, and can include multiple rigid or semi-rigid components pivotally secured to one another to allow the transition of the implant frame 300 between the undeployed configuration and the deployed configuration associated with the delivery configuration and the functional configuration of the implantable body armor 10, respectively.

In some implementations, the implant frame 300 can be a self-deployable implant frame 300 configured to change from the undeployed configuration to the deployed configuration. The self-deployable implant frame 300 can be configured to enable an autonomous transition, i.e., operable without the assistance of external intervention such as the delivery system, of the implantable body armor 10 from the delivery configuration to the functional configuration when the implantable body armor 10 has been delivered at the implantation site. In such implementations, the implant frame 300 can include one or more resilient members, which can be integrated within the implant frame 300, the resilient members being configured to bias the implant frame 300 into the functional configuration.

In some implementations, the implant frame 300 can be made of a material that enables the implant frame 300 to modify its configuration over time. In some implementations, the implant frame 300 can be made of an implant frame material that is bioabsorbable, the implant frame material having properties which can vary to modify one or more characteristics of the implant frame 300 within a period of time, for instance 60 days, following an implantation of the implantable body armor 10 at the implantation site. The absorbable nature of the implant frame 300 can stem from its susceptibility to breakdown, decomposition, degradation, resorption and/or dissolution, under the action of biological processes, such as enzymatic reaction and/or hydrolysis. In some implementations, the implant frame 300 can be made of an aliphatic polyester or a combination of aliphatic polyesters, or can include one or more portions made of an aliphatic polyester or a combination of aliphatic polyesters. The aliphatic polyester can be a synthetic aliphatic polyester. Examples of aliphatic polyester include polylactic acid, polyglycolic acid, polylactic-co-glycolic acid, polycaprolactone, and polydioxanone.

In implementations where the implantable body armor 10 comprises the fixation mechanism for securing the implantable body armor 10 at the implantation site within the cavity of the human body, the implant frame 300 can similarly include a fixation mechanism engaging feature similar to the fixation mechanism engaging feature 134 of the flexible panel 100 described above and configured to be engaged by the fixation mechanism 132 during the securing of the flexible panel 100 to the bone or soft tissue.

### Methods for Delivering an Implantable Body Armor

A method for delivering an implantable body armor at an implantation site will now be described in further detail.

The method can include delivering the implantable body armor to an implantation site in a delivery configuration, and subsequently transitioning the implantable body armor from the delivery configuration to a functional configuration once the implantable body armor is positioned at the implantation site to provide an impact resistance to an internal organ. The delivery configuration can be different than the functional configuration. In some implementations, the delivery configuration can facilitate a navigation of the implantable body armor via a delivery path which extends from the outside of the human body to the implantation site. For instance, when the implantable body armor is delivered through a percutaneous incision, the delivery configuration of the implantable body armor can therefore enable a health care provider in charge of the implantation of the implantable body armor to use a minimally invasive approach by passing the flexible panel through a smaller percutaneous incision than if the implantable body armor was not configurable in a delivery configuration, while reducing potential trauma that could occur to surrounding tissues during delivery of the implantable body armor to the implantation site.

The implantable body armor can include a flexible panel including layers of woven fibers forming an impact-resistant structure configured to absorb impact energy, as described above. In particular, the flexible panel can include any number of superposed layers of woven fibers that is sufficient to provide an impact-resistant structure while retaining a desired flexibility for implantation in the human body.

Various techniques can be used to deliver the implantable body armor to the implantation site via the delivery path. For instance, in some implementations, the implantable body armor can be delivered through open surgery or via an endoscopic procedure while in the delivery configuration. The endoscopic procedure can be performed using, for instance, third space endoscopy, videoscopic endoscopy, a thoracoscopic procedure or a laparoscopic procedure, while the implantable body armor is in the delivery configuration.

In some implementations, the implantable body armor can be delivered through open surgery or endoscopically to a subcutaneous implantation site or a submuscular implantation site via a percutaneous incision performed in proximity to the desired implantation site.

In other implementations, the implantable body armor can be delivered through open surgery or endoscopically to an implantation site within a cavity of the human body. For instance, when the desired implantation site is located within the abdominopelvic cavity and an endoscopic procedure is indicated, the implantable body armor can be delivered by a laparoscopic delivery along a delivery path extending through the abdominal wall and optionally through the peritoneum.

Alternatively, in other implementations, when the implantation site is located within the thoracic cage and an endoscopic procedure is indicated, the implantable body armor can be delivered by thoracoscopic delivery. In such implementations, the implantable body armor can be delivered by thoracoscopic delivery along a delivery path extending through an intercostal space of the thoracic cage for delivery into the thoracic cavity. It is to be understood that, in other implementations, the implantable body armor can be delivered through open surgery or endoscopically following another delivery path in accordance with a different desired implantation site.

In some implementations, the delivery of the implantable body armor can be achieved through use of a delivery system. In such implementations, an attachment feature of the implantable body armor can be releasably connected to the delivery system. More specifically, the attachment feature of the implantable body armor can be releasably connected to a connector of the delivery system to allow a delivery of the implantable body armor to the desired implantation site. An example of a suitable delivery system includes a delivery catheter. The delivery catheter can include the connector as described above.

In some implementations, the attachment feature can enable a deployment of the flexible panel into the functional configuration. As stated above, in some implementations, the implantable body armor can include a self-deployable implant frame configured to enable an autonomous transition of the implantable body armor from the delivery configuration to the functional configuration when the implantable body armor has been delivered at the implantation site. In other words, the implantable body armor can be self-deployable and thus operable without assistance, or without significant assistance, from an external intervention. In such implementations, the release of the attachment feature from the connector of the delivery system can trigger the autonomous transition of the implantable body armor into the functional configuration.

Once the implantable body armor is delivered to the implantation site, the implantable body armor can be fastened to one or more of a soft tissue and a hard tissue in the vicinity of the implantation site including, for instance, an internal surface of a thoracic cage. The fastening of the implantable body armor can be achieved using a fixation mechanism configured to engage the flexible panel with a hard tissue and/or a soft tissue located in the vicinity of the implantation site, as described above.

In some implementations, when the desired implantation site of the implantable body armor includes an anatomic potential space having a dynamic volume, the delivery of the implantable body armor can include an expansion of the anatomic potential space to accommodate the insertion of the implantable body armor therein, as well as to provide additional workspace to perform the transition of the implantable body armor from the delivery configuration to the functional configuration, and to fasten the implantable body armor to an adjoining tissue. Examples of anatomic potential spaces suitable for providing an implantation site of the implantable body armor are specified above and include, for instance, the pleural cavity surrounding the lungs and the peritoneal cavity surrounding the abdominal organs. In some implementations, the expansion of the anatomic potential space can be achieved by insufflation of the anatomic potential space using a gas, such as carbon dioxide. In other implementations, the expansion of the anatomic potential space can be achieved by a mechanical means, such as forceps or a mechanical expander.

When the implantable body armor is implanted in the functional configuration at the implantation site, a growth of soft tissue and/or hard tissue can be promoted within interstices defined by adjacent fibers of the woven fibers of the layers of woven fibers. A growth of said tissues within the interstices can contribute to an integration of the implantable body armor into an adjoining tissue in the vicinity of the implantation site. More specifically, the interstices can provide a space suitable for the migration and proliferation of the adjoining biological tissue, thereby promoting the growth of said tissue, as described above.

In some implementations, the method can further include depositing a biocompatible coating onto one or more of the adjacent fibers of the woven fibers of the layers of woven fibers defining the interstices. In some implementations, the biocompatible coating can include a bioactive agent such as a cell growth promoter configured to induce a growth of cells within the interstices of the woven fibers to further promote a growth of soft tissue or hard tissue therein.

Several alternative implementations and examples have been described and illustrated herein. The implementations of the technology described above are intended to be exemplary only. A person of ordinary skill in the art would appreciate the features of the individual implementations, and the possible combinations and variations of the components. A person of ordinary skill in the art would further appreciate that any of the implementations could be provided in any combination with the other implementations disclosed herein. It is understood that the technology may be embodied in other specific forms without departing from the central characteristics thereof. The present implementations and examples, therefore, are to be considered in all respects as illustrative and not restrictive, and the technology is not to be limited to the details given herein. Accordingly, while the specific implementations have been illustrated and described, numerous modifications come to mind.

1. An implantable body armor for placement at an implantation site within a ventral cavity of a human body, the implantable body armor comprising:
a flexible panel having an intercostal surface and a cavity surface opposite the intercostal surface, the flexible panel comprising:
layers of woven fibers forming an impact-resistant structure configured to absorb impact energy.

2. The implantable body armor of claim 1, wherein the cavity surface has a cavity surface roughness that is higher than an intercostal surface roughness of the intercostal surface.

3. The implantable body armor of claim 1, further comprising an implant frame engageable with the flexible panel and positionable around an outer periphery of the flexible panel.

4. The implantable body armor of claim 3, wherein the implant frame is a self-deployable implant frame configured to transition the implantable body armor from a delivery configuration to a functional configuration once the implantable body armor is delivered at the implantation site.

5. The implantable body armor of claim 4, wherein the implant frame is made of an implant frame material, the implant frame material being bioabsorbable.

6. The implantable body armor of claim 1, further comprising a fixation mechanism to engage the flexible panel with a hard tissue or a soft tissue located in the vicinity of the implantation site.

7. The implantable body armor of claim 6, wherein the fixation mechanism is configured to engage the flexible panel with an internal surface of a thoracic cage.

8. The implantable body armor of claim 6, wherein the fixation mechanism is made of a fixation mechanism material, the fixation mechanism material being bioabsorbable.

9. The implantable body armor of claim 1, wherein adjacent fibers of the layers of woven fibers define interstices sized to promote a growth of tissue therebetween to contribute to an integration of the flexible panel into an adjoining tissue.

10. The implantable body armor of claim 9, wherein an outermost layer of the layers of woven fibers comprises:
a first interstice zone defining first zone interstices having a first size selected to promote a growth of tissue therebetween; and
a second interstice zone defining second zone interstices having a second size to prevent a growth of tissue therebetween, the second size being smaller than the first size.

11. The implantable body armor of claim 1, wherein fibers of the layers of woven fibers comprise aramid fibers.

12. The implantable body armor of claim 11, wherein at least one layer of the layers of woven fibers further comprises additional fibers.

13. The implantable body armor of claim 12, wherein the additional fibers comprise bioabsorbable fibers absorbable at physiological conditions.

14. The implantable body armor of claim 12, wherein the additional fibers have hydrophobic properties to prevent bacterial adhesion in an interstice defined between adjacent fibers of the layers of woven fibers.

15. The implantable body armor of claim 12, wherein at least a portion of the additional fibers comprises a bioactive agent having therapeutic properties.

16. The implantable body armor of claim 1,
further comprising a panel cover at least partially covering the flexible panel, the panel cover being made of a biocompatible material, or
wherein outermost layers of the layers of woven fibers comprise a biocompatible coating provided onto an external surface thereof.

17. The implantable body armor of claim 16, wherein the biocompatible material or the biocompatible coating comprises a bioactive agent having therapeutic properties.

18. The implantable body armor of claim 16, wherein the flexible panel comprises a reinforcement zone provided at a selected region of the flexible panel to provide an additional impact-resistant structure.

19. The implantable body armor of claim 18, wherein the reinforcement zone is engaged with the cavity surface or the outer surface of the flexible panel.

20. The implantable body armor of claim 18, wherein the reinforcement zone is provided between adjacent ones of the layers of woven fibers of the flexible panel.

21. An implantable body armor for placement at an implantation site within a human body, the implantable body armor comprising:
a flexible panel comprising:
layers of woven fibers forming an impact-resistant structure configured to absorb impact energy;
wherein the implantable body armor is configurable between a delivery configuration and a functional configuration;
wherein when in the delivery configuration, the flexible panel of the implantable body armor has a resulting external surface area that is smaller than when in the functional configuration to facilitate a delivery of the implantable body armor to the implantation site.
